(19) United States
(12) Patent Application Publication
(10) Pub. No.: US 2007/0017506 A1
(43) Pub. Date: Jan. 25, 2007

(54) Dispensing apparatus

(76) Inventors: Phillip Bell, Raleigh, NC (US); John Popow, Cary, NC (US)

Correspondence Address: Dennis C. Rodgers, 1850 M. Street N.W., Suite 800, Washington, DC, 20036, US

(21) Appl. No.: 10/575,866

(22) PCT Filed: Jun. 4, 2004
(86) PCT No.: PCT/GB04/02315

§ 371 Date: Apr. 17, 2006

(30) Foreign Application Priority Data
Oct. 16, 2003 (GB) 0324278.1

(51) Int. Cl. A61M 11/00; A61M 15/00

(52) U.S. Cl. 128/200.23; 128/203.15

(57) ABSTRACT

A dispenser (1) comprising a housing (3), a pressure sensor (22), processing means (23) and a display means (27), the housing being shaped for receiving, in use, a dispensing container (5) of the type containing medicament and having valve means for dispensing the medicament in metered volume doses, wherein, in use, the pressure sensor is capable of detecting a pressure signature produced on dispensation of medicament from the dispensing container, wherein the pressure sensor is operatively connected to the processing means for relaying signals indicative of the pressure signature for processing by the processing means, the processing means being programmed to analyse said signals and compare said signals against one or more data sets containing data indicative of one or more control pressure signatures, the processing means being programmed to use a result of said comparison to detect the quantity of medicament dispensed compared to an intended volume of the metered dose volume.

### DISPENSING APPARATUS

The present invention relates to a dispenser for use with a dispensing container which contains a product to be dispensed in metered doses. One example, is a metered dose inhaler which includes a pressurised dispensing container containing a medicament.

It is known to dispense products, including medicaments, in metered volume doses. Typically, dispensers for metered dispensation include metering valves which are designed to dispense a known volume of the product in a consistent and reproducible manner. This is important to ensure that each dose dispenses the correct volume of product. However, it is known that the manner in which the dispenser is operated by a user can affect the volume accuracy of doses dispensed. For example, if the metering valve of the dispenser is not fully actuated then a partial dose may be dispensed instead of a full dose. Also, if the dispenser is actuated in a non-standard orientation, e.g. upside-down, a partial dose may be dispensed and/or re-filling of the metering valve may be impeded leading to the a subsequent partial dose being dispensed.

It is also desirable for a user to be able to ascertain the remaining number of doses in the dispenser and/or the number of doses already administered. This can ensure that a replacement dispenser is obtained in a timely manner and also ensure that a dispenser is not used after the active product has been exhausted (it is common for the dispenser to continue to dispense ancillary components, such as propellant, after the active component of the product has become exhausted). Dosage counters may also be used to check user compliance with intended dosage regimes. It has been attempted to include dosage counters in such dispensers to meet these needs. However, known dosage counters can inaccurately indicate the quantity of product dispensed or remaining in the dispensing container if a number of partial doses are dispensed due to the problems mentioned above.

The present invention provides a dispenser comprising a housing, a pressure sensor, processing means and a display means, the housing being shaped for receiving, in use, a dispensing container of the type containing medicament and having valve means for dispensing the medicament in metered volume doses, wherein, in use, the pressure sensor is capable of detecting a pressure signature produced on dispensation of medicament from the dispensing container, wherein the pressure sensor is operatively connected to the processing means for relaying signals indicative of the pressure signature for processing by the processing means, the processing means being programmed to analyse said signals and compare said signals against one or more data sets containing data indicative of one or more control pressure signatures, the processing means being programmed to use a result of said comparison to detect the quantity of medicament dispensed compared to an intended volume of the metered dose volume.

The comparison between the acoustic signature and the dose delivered may be developed through a series of tests analysing the acoustic signature for the delivery of different amounts of medicament (for example, by filling the metering chamber to different levels). Data derived from the measured parameters may then be stored for use by the processing means to perform the comparison when the dispenser is actuated.

Measurement and analysis of the pressure signature and comparison with one or more control pressure signatures allows for detection of dispensation of a partial dose. Estimation of the proportion of the dose dispensed may also be made.

Acoustic signatures which are not dose related, for example those caused by background noise, are preferably filtered out by the system.

Preferably, acoustic signatures falling within the amplitude and frequency associated with dose delivery are used to initiate automatically the correlation procedure.

Preferably the processing means is programmed to output a first signal to the display means when said comparison indicates that the quantity of medicament dispensed substantially matches an intended volume of said metered volume dose to thereby update the display means to reflect that a metered volume dose has been dispensed.

Preferably, the processing means is programmed to produce a second signal when said comparison indicates that the quantity of medicament dispensed is substantially less than an intended volume of said metered volume dose.

Preferably, the second signal contains data indicative of the proportion of the intended volume of said metered volume dose actually dispensed. optionally, the second signal, or a derivative thereof, is used to update the display means to reflect that a proportion of a metered volume dose has been dispensed. optionally, the second signal, or a derivative thereof, is used to produce an alert to instruct a user to administer a further dose.

In this way if the dispensed volume is significantly below the intended dosage so as not to achieve a desired result the apparatus may prompt the user to immediately take a further dose. However, if the dispensed volume is only marginally below the intended dosage a further dosage would preferably not be prompted to prevent too great a quantity being administered.

Preferably the processing means contains an accumulated volume variable indicative of the accumulated volume of medicament dispensed by the dispensing container. Typically, the second signal, or a derivative thereof, is used to update the accumulated volume variable. optionally, the accumulated volume variable is used to update the display means to indicate the quantity of medicament dispensed from the dispensing container and or the quantity of medicament remaining in the dispensing container.

An approximation of the accumulated quantity of product remaining in the dispensing container or dispensed therefrom may thus be made. As the amount of medicament dispensed is more accurately monitored, the dispensing container may be safely used nearer its exhaustion point.

The processing means may analyse one or more of the frequency, duration, area, rising slope, falling slope and amplitude of the pressure signature.

In one embodiment, the processing means applies a band-pass filter to the pressure signature.

The processing means may select a signature envelope for further signal processing.

The processing means may apply a notch filter to the pressure signature to slice the signature into discrete segments of equal time duration. The processing means may then compare the number of signal-containing segments with a control number derived from the one or more data sets.

In one embodiment the pressure sensor is an acoustic sensor and the pressure signature is an acoustic signature.

Alternatively, the pressure sensor is selected from the group consisting of a vibration sensor, a strain sensor, a compression sensor, a deflection sensor and a flow sensor.

The acoustic sensor may be a microphone. The microphone may a micro-electro-magnetic microphone.

The acoustic sensor may comprise piezoelectric material.

In one embodiment the pressure sensor is contacted, in use, by the dispensed medicament . The pressure sensor may be deflectable by the impact of medicament during dispensation to thereby produce the pressure signature. The pressure sensor may comprise a piezoelectric strip in the form of a cantilever. Alternatively, the pressure sensor may comprise a piezoelectric surface in the form of a drum-skin.

The pressure sensor may be isolated, in use, from contact with the dispensed medicament. Optionally, the pressure sensor is located in acoustic contact with an acoustic chamber.

In one embodiment, the dispensing container received, in use, in the housing is of the type comprising a valve stem through which the medicament is dispensed, the housing further comprising a stem block for receiving said valve stem, the stem block comprising a conduit for directing medicament dispensed through said valve stem towards an outlet of the dispenser, wherein the acoustic chamber is located in acoustic contact with the conduit.

The acoustic chamber is preferably sealed to prevent the ingress of fluid or particles which might otherwise affect the measured acoustic signature and result in inaccurate measurements and/or correlations. Furthermore, locating the sensor in a chamber helps to minimise or obviate the effect on the air/drug flow path whilst facilitating a high degree of accuracy and reliability. The sensor is also protected from damage when it is located in a sealed chamber.

The acoustic chamber may be located within the stem block. Alternatively, the pressure sensor may be located on an external surface of the stem block. The pressure sensor may forms one wall of the acoustic chamber.

In another embodiment the dispenser comprises a pyroelectric sensor for detecting temperature changes within the housing during dispensation of medicament. The pyroelectric sensor is operatively connected to the processing means for relaying signals indicative of the temperature for processing by the processing means. During dispensation from the pressurised dispensing container there are appreciable temperature drops within the housing, in particular in the region of the stem block, caused by the rapid expansion of the volatile propellant. This temperature drop may advantageously be used in combination with the use of a pressure sensor to exclude false positive counts. The processing means is programmed to exclude signals resembling the data set of a dose dispensation if there is no associated temperature drop.

Preferably, the processing means is programmed to analyse said temperature signals and compare said signals against one or more data sets containing data indicative of one or more control temperature signatures, the processing means being programmed to use a result of said comparison to detect the actuation of the dispensing container.

The acoustic sensor and/or processing means may be fixedly mounted on the housing of the dispenser. Alternatively, the sensor and/or processing means may be detachably mounted on the dispensing container to enable the dispensing container to be used with conventional dispensing apparatus. A further alternative is to detachably mount the sensor and/or processing means on the housing of the dispenser.

The display may be a thin film liquid crystal display. The display may be detachably or fixedly mounted on the housing of the dispenser.

The valve incorporated in the dispenser may be, for example, for use in a pharmaceutical dispensing device, such as, for example, a pulmonary, nasal, or sub-lingual delivery device. A preferred use of the valve is in a pharmaceutical metered dose aerosol inhaler device. The term pharmaceutical as used herein is intended to encompass any pharmaceutical, compound, composition, medicament, agent or product which can be delivered or administered to a human being or animal, for example pharmaceuticals, drugs, biological and medicinal products. Examples include antiallergics, analgesics, bronchodilators, antihistamines, therapeutic proteins and peptides, antitussives, anginal preparations, antibiotics, anti-inflammatory preparations, hormones, or sulfonamides, such as, for example, a vasoconstrictive amine, an enzyme, an alkaloid, or a steroid, including combinations of two or more thereof. In particular, examples include isoproterenol [alpha-(isopropylaminomethyl) protocatechuyl alcohol], phenylephrine, phenylpropanolamine, glucagon, adrenochrome, trypsin, epinephrine, ephedrine, narcotine, codeine, atropine, heparin, morphine, dihydromorphinone, ergotamine, scopolamine, methapyrilene, cyanocobalamin, terbutaline, rimiterol, salbutamol, flunisolide, colchicine, pirbuterol, beclomethasone, orciprenaline, fentanyl, and diamorphine, streptomycin, penicillin, procaine penicillin, tetracycline, chlorotetracycline and hydroxytetracycline, adrenocorticotropic hormone and adrenocortical hormones, such as cortisone, hydrocortisone, hydrocortisone acetate and prednisolone, insulin, cromolyn sodium, and mometasone, including combinations of two or more thereof.

The pharmaceutical may be used as either the free base or as one or more salts conventional in the art, such as, for example, acetate, benzenesulphonate, benzoate, bircarbonate, bitartrate, bromide, calcium edetate, camsylate, carbonate, chloride, citrate, dihydrochloride, edetate, edisylate, estolate, esylate, fumarate, fluceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylbromide, methylnitrate, methylsulphate, mucate, napsylate, nitrate, pamoate, (embonate), pantothenate, phosphate, diphosphate, polygalacturonate, salicylate, stearate, subacetate, succinate, sulphate, tannate, tartrate, and triethiodide, including combinations of two or more thereof. Cationic salts may also be used, for example the alkali metals, e.g. Na and K, and ammonium salts and salts of amines known in the art to be pharmaceutically acceptable, for example glycine, ethylene diamine, choline, diethanolamine, triethanolamine, octadecylamine, diethylamine, triethylamine, 1-amino-2-propanol-amino-2-(hydroxymethyl)propane-1,3-diol, and 1-(3,4-dihydroxyphenyl)-2 isopropylaminoethanol.

The pharmaceutical will typically be one which is suitable for inhalation and may be provided in any suitable form for this purpose, for example as a solution or powder suspension in a solvent or carrier liquid, for example ethanol, or isopropyl alcohol. Typical propellants are HFA134a, HFA227 and di-methyl ether.

The pharmaceutical may, for example, be one which is suitable for the treatment of asthma. Examples include salbutamol, beclomethasone, salmeterol, fluticasone, formoterol, terbutaline, sodium chromoglycate, budesonide and flunisolide, and physiologically acceptable salts (for example salbutamol sulphate, salmeterol xinafoate, fluticasone propionate, beclomethasone dipropionate, and terbutaline sulphate), solvates and esters, including combinations of two or more thereof. Individual isomers such as, for example, R-salbutamol, may also be used. As will be appreciated, the pharmaceutical may comprise of one or more active ingredients, an example of which is flutiform, and may optionally be provided together with a suitable carrier, for example a liquid carrier. One or more surfactants may be included if desired.

The seals and gaskets of the valve may be formed from any suitable material having acceptable performance characteristics. Preferred examples include nitrile, EPDM and other thermoplastic elastomers, butyl and neoprene.

Other rigid components of the metering valve, such as the valve body, chamber body and valve stem may be formed, for example, from polyester, nylon, acetal or similar. Alternative materials for the rigid components include stainless steel, ceramics and glass.

The housing of the dispenser including components such as the stem block and mouthpiece may be formed, for example, from polyethylene (PE), polypropylene (PP), polycarbonate (PC), polybutylene terephthalate (PBT), acrylonitrile-butadiene-styrene (ABS), or similar engineering plastics.

Preferred embodiments of the present invention will now be described, by way of example only, with reference to the accompanying drawings in which:

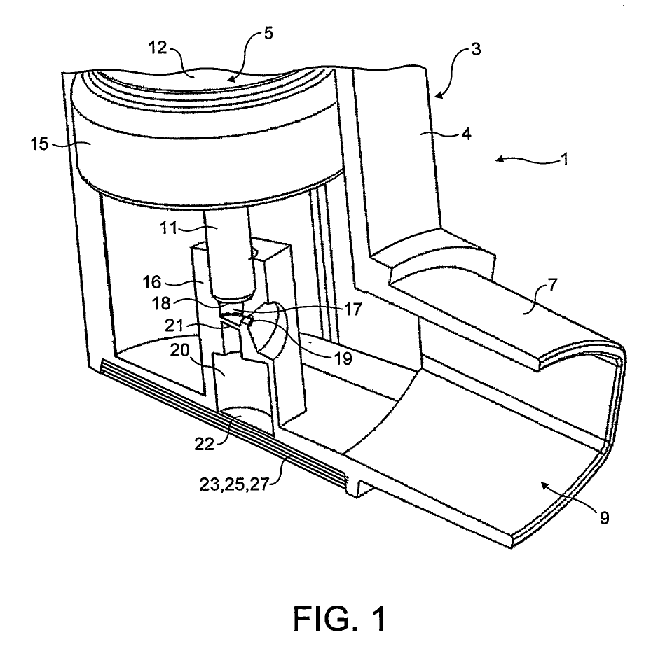

FIG. 1 shows a partial cross-sectional perspective view of a dispenser in accordance with a first embodiment of the present invention;

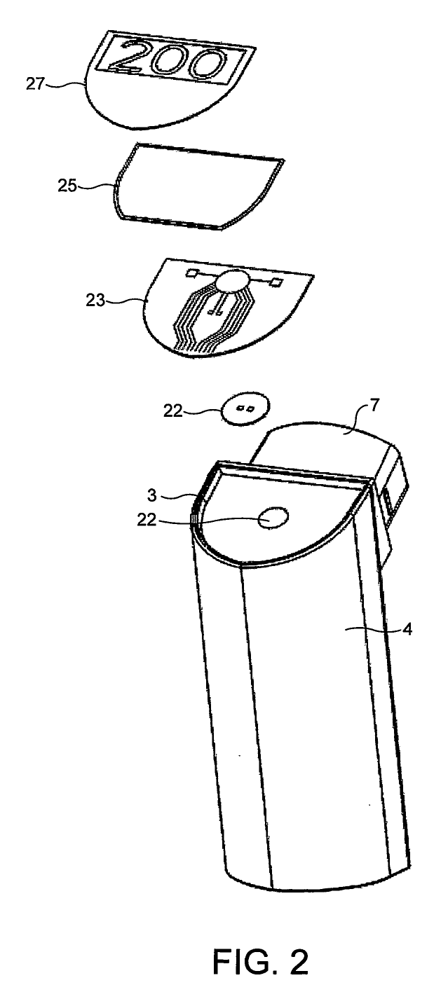

FIG. 2 shows an exploded perspective view of the underside of the dispenser shown in FIG. 1;

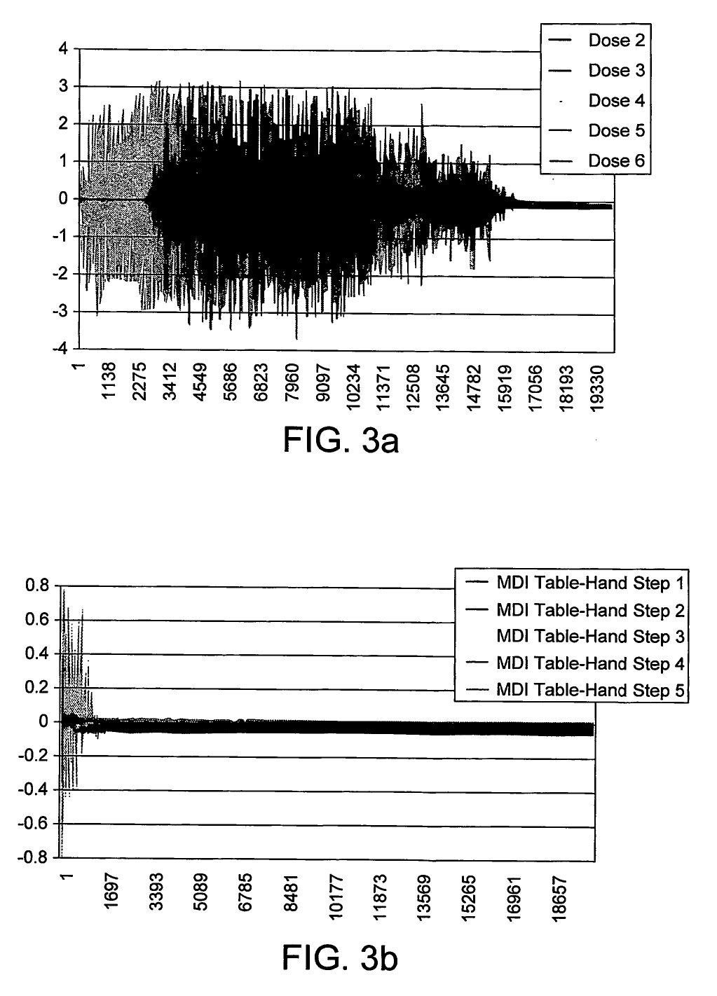

FIG. 3a shows the measured acoustic signature for a five typical actuations of the dispenser;

FIGS. 3b to 3f show the measured acoustic signatures of a variety of typical background noises;

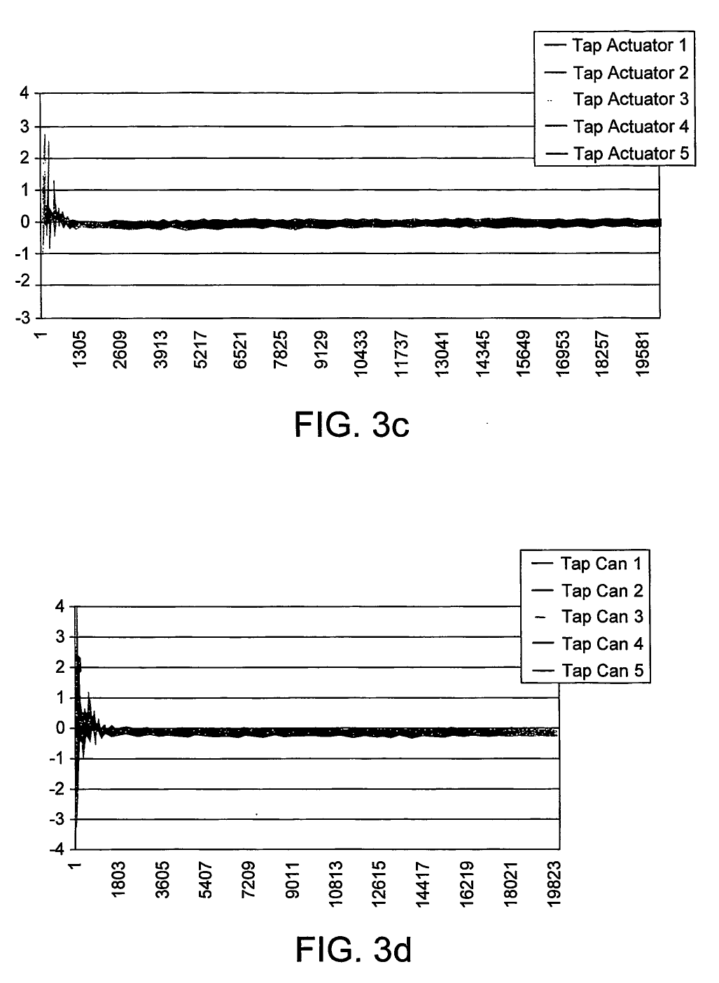

FIGS. 4a and 4b illustrate schematically the signal processing applied to the acoustic signature;

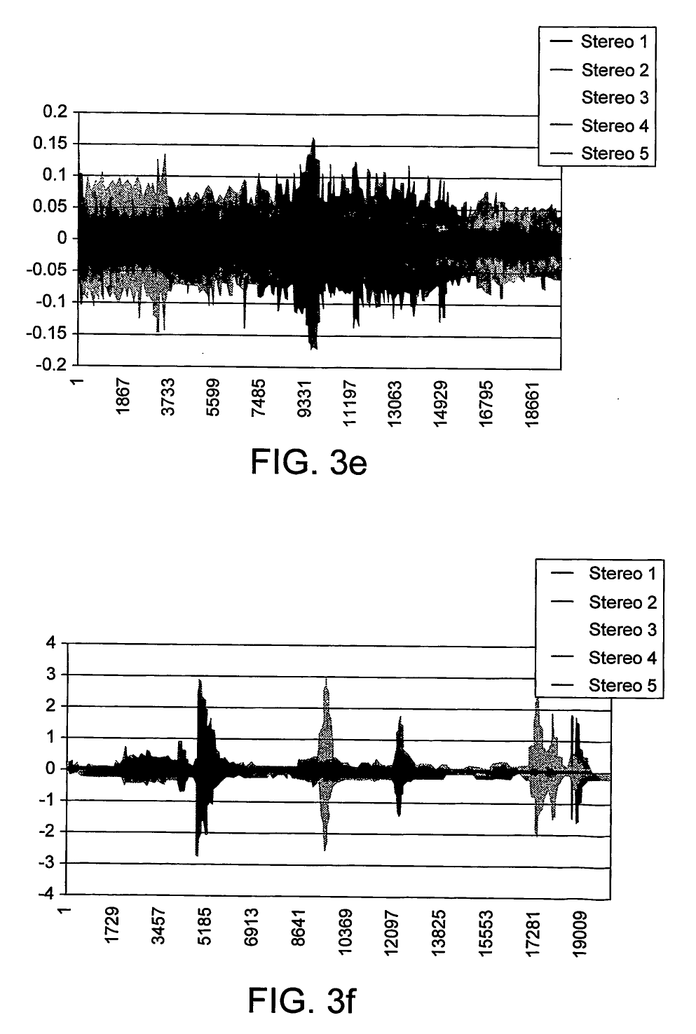

FIG. 5 shows a perspective view of a second embodiment of the present invention;

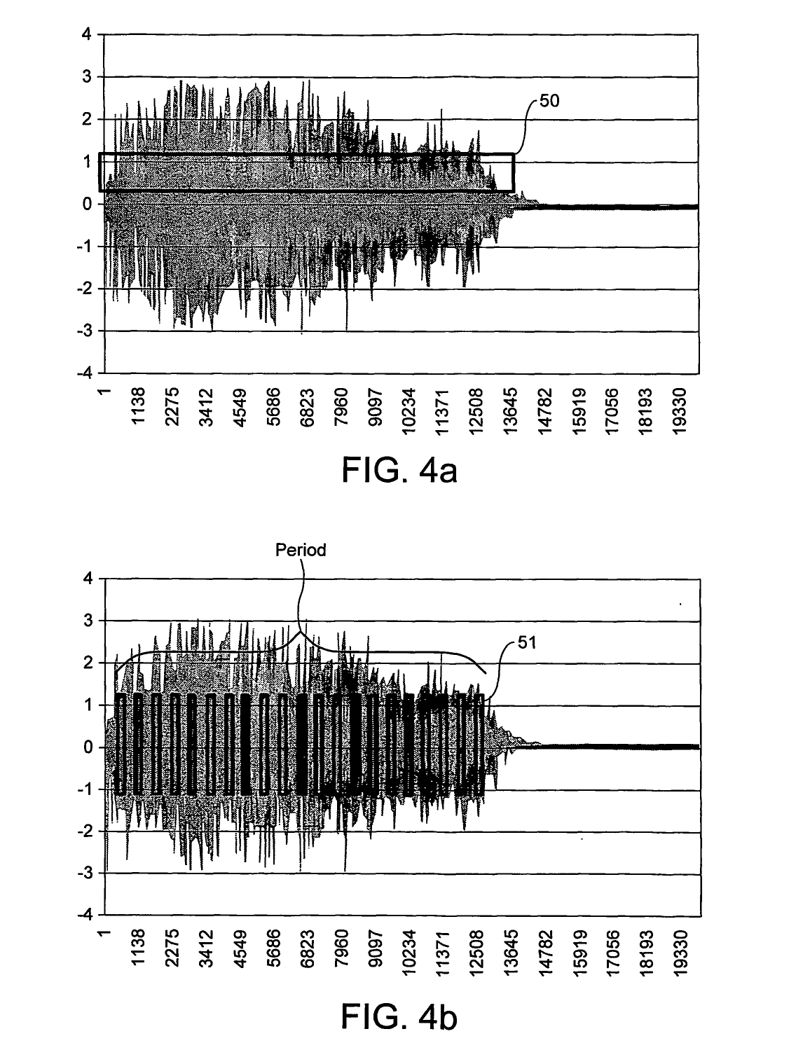

FIG. 6 shows a partial cross-sectional perspective view of a dispenser in accordance with a third embodiment of the present invention;

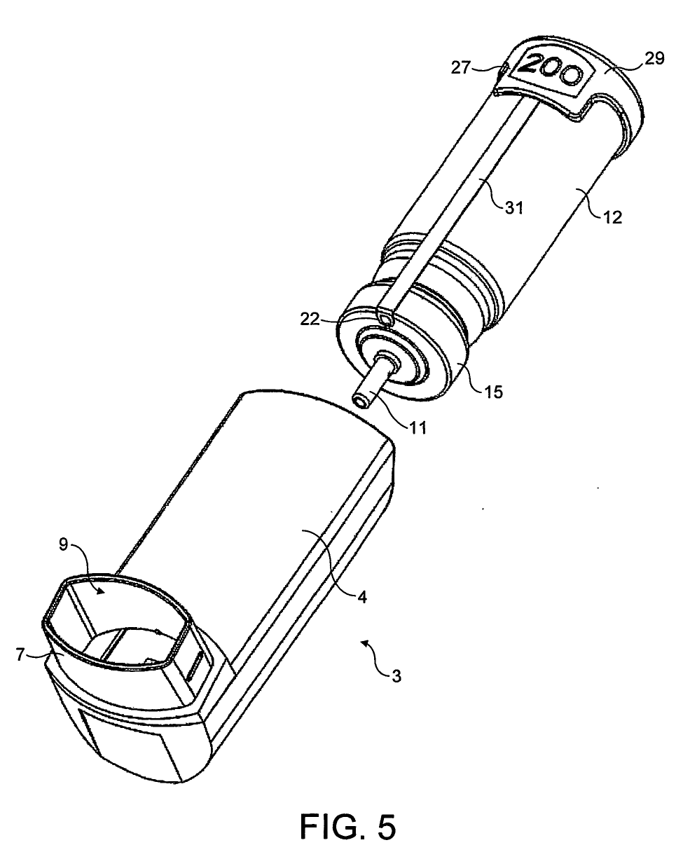

FIG. 7 shows a partial cross-sectional perspective view of a dispenser in accordance with a fourth embodiment of the present invention;

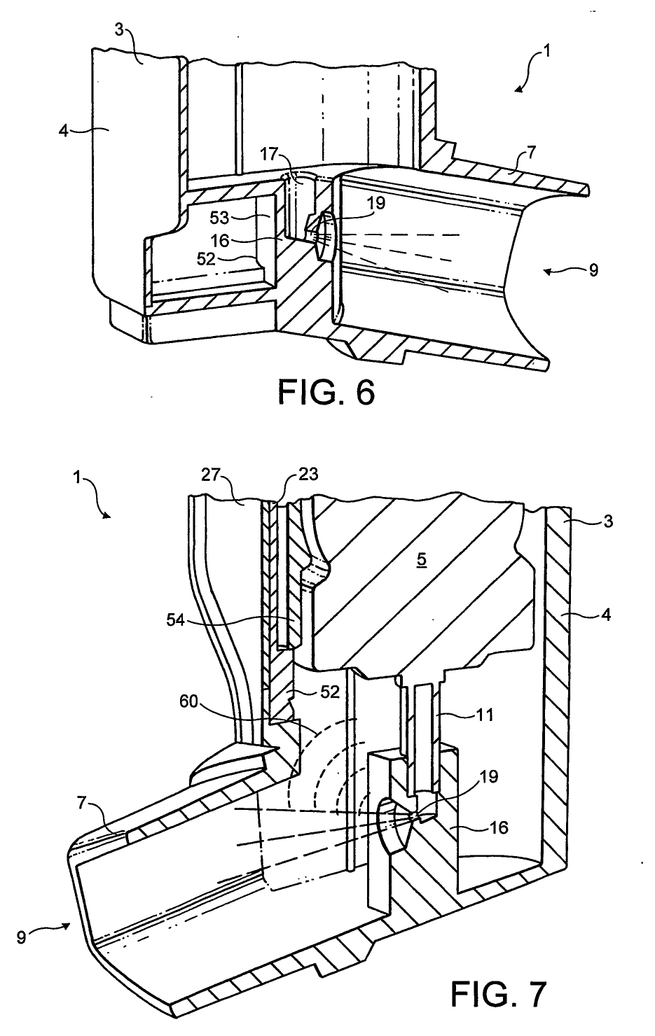

FIG. 8 shows a partial cross-sectional view of a dispenser in accordance with a fifth embodiment of the present invention; and

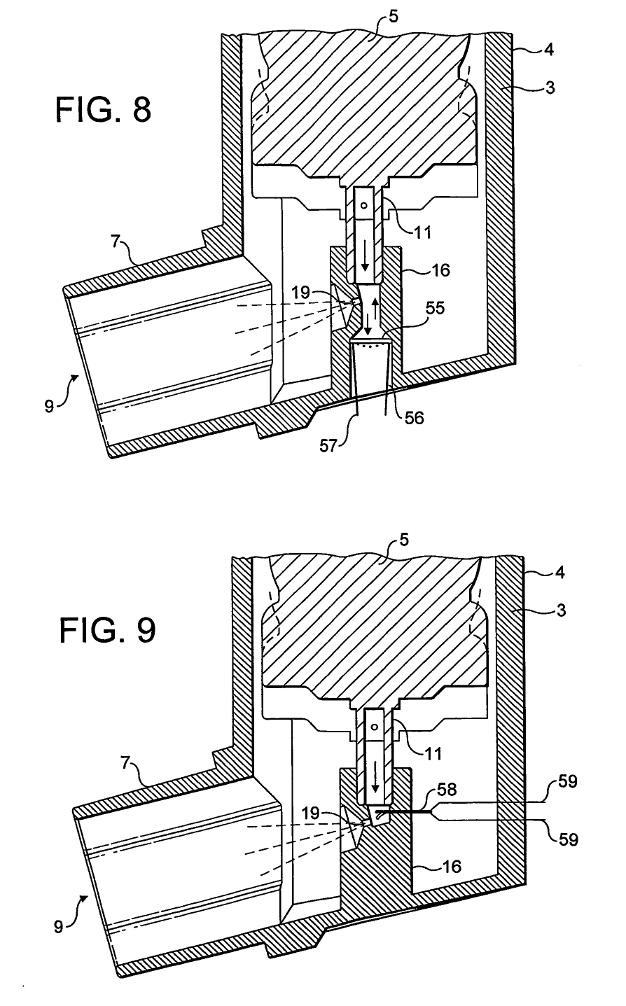

FIG. 9 shows a partial cross-sectional view of a dispenser in accordance with a sixth embodiment of the present invention.

A dispenser 1, shown by way of example as a metered dose inhaler, in accordance with a first embodiment of the present invention is shown in FIGS. 1 and 2. The metered dose inhaler comprises the combination of a housing 3 (commonly known as an actuator) and a pressurised dispensing container 5 containing product to be dispensed.

The pressurised dispensing container 5 is of conventional construction known in the art and comprises a metering valve located to seal an open end of a canister 12. The metering valve is held in position on the canister 12 by means of a crimped ferrule 15. A valve stem 11 of the metering valve extends from the metering valve as shown in FIG. 1. The metering valve comprises an internal metering chamber which permits a fixed volume of product to be dispensed on each actuation of the metering valve.

The housing 3 is shaped and configured to receive the pressurised dispensing container 5 and permits the pressurised dispensing container 5 to be operated manually by a user. The housing 3 comprises a tubular body portion 4 having an opening (not shown) at an upper end through which the pressurised dispensing container 5 is received. An integrally formed mouthpiece 7 is provided at a lower end of the body portion 4 and comprises an opening 9 at a distal end.

The lower end of the housing 3 also comprises a stem block 16 in which the valve stem 11 of the pressurised dispensing container 5 is received in a sliding manner on insertion of the pressurised dispensing container 5 into the housing 3. The stem block 16 comprises an upper bore 17 in which a distal end 18 of the valve stem 11 can fit. A lower end of the upper bore 17 communicates with an orifice 19 of relatively small diameter which is directed towards the opening 9 of the mouthpiece 7.

In use, downward displacement of the pressurised dispensing container 5 towards the lower end of the housing 5 causes the valve stem 11 to be displaced relative to the remainder of the metering valve resulting in actuation of the metering valve and dispensation of a metered volume of product. For propellant-driven dispensers, dispensation occurs due to rapid volatilisation and expansion of liquefied propellant within the metering chamber of the metering valve. Volatilisation and expansion of the propellant creates a rapid and large pressure differential between the metering valve and atmosphere. As a result the product is rapidly discharged to atmosphere. Operation of the metering valve is conventional and well known in the art and will not be described in detail here. The product is dispensed out of the distal end 18 of the valve stem 11 into the upper bore 17. Due to the driving force of the expanding propellant, the product is forced from upper bore 17 through the orifice 19 where the product is atomised and then discharged through mouthpiece 7 for inhalation.

According to the present invention, the stem block 16 further comprises a pressure chamber in the form of an acoustic chamber 20 formed below the upper bore 17 and orifice 19 as shown in FIG. 1. The acoustic chamber 20 is separated from the upper bore 17 by a partition 21 at an upper end of the acoustic chamber 20 so that the acoustic chamber is physically isolated from contact with the product during dispensation. The acoustic chamber 20 is, however, in acoustic contact with the upper bore 17 and orifice 19. In other words, acoustically-produced vibrations in the upper bore 17 and orifice 19 are transmitted into the acoustic chamber 20. Transmittal of acoustic signals is primarily via partition 21 although some transmittal may also occur via the remaining structure of the stem block 16.

A lower end of the acoustic chamber 20 is closed off by a sensor 22 such that the acoustic chamber 20 defines a closed volume isolated from atmosphere. In particular ingress of product from the upper bore 17 and moisture and contaminants from the exterior of the dispenser 1 is prevented. The acoustic chamber 20 acts to amplify and enhance the acoustic signals picked up from the upper bore 17 and orifice 19. The exact shape and volume of the acoustic chamber 20 may be varied to suit the characteristics of the dispenser 1. In the present example the acoustic chamber 20 is tubular and comprises two distinct regions of differing diameter which is believed to act to aid amplification of acoustic signals within the acoustic chamber 20.

The sensor 22 located at the lower end of the acoustic chamber 20 is in the form of a thin laminar of piezoelectric material. One example of a suitable material is PVDF. The sensor 22 is operatively connected to an integrated circuit board 23. The circuit board 23 has a processor and a memory provided thereon and is connected to a thin film battery 25 and a thin film liquid crystal display 27. The sensor 22 is mounted directly to the base of the housing 3, the circuit board 23 is then mounted on the sensor 22 with the battery 25 being mounted to the circuit board 23. Finally the display 27 is mounted on to the battery 25. Thus, a sandwiched configuration is produced comprising the housing base, sensor 22, circuit board 23, battery 25 and display 27. The circuit board 23 is electrically connected to the battery 25, sensor 22 and display 27. The processor of the circuit board provides power to the display 27. The memory stores in a read-only memory, such as an EPROM, pre-programmed information required for processing of signals received from the sensor 22. The memory also comprises a writable and readable form of memory capable of storing received data from the processor during use of the dispenser 1. The processor processes signals received from the sensor 22 as described below.

As described above, actuation of the dispenser 1 is achieved by compressing the valve stem 11. The dispensation of the product produces pressure changes resulting in vibrations within the dispenser 1 as the product is driven first from the metering valve into the upper bore 17 of the stem block 16 and then through the orifice 19 into the mouthpiece. The vibrations occur across a broad range of frequencies. In the present embodiment, frequencies which may be termed ‘acoustic’ are of primary interest. Generally, acoustic frequencies are defined as those frequencies detectable to the human ear.

The vibrations are transmitted into the acoustic chamber 20 where they reverberate. The dimensions of the acoustic chamber 20 may be chosen to cause selected frequencies to resonate and thus be amplified by superposition. Likewise certain frequencies may be diminished in amplitude by careful selection of the chamber dimensions. This can allow unwanted frequencies to be reduced.

The sensor 22 is in turn vibrated by the acoustic vibrations causing the thin film of piezoelectric material to flex. Due to the physical properties of the piezoelectric material the flexure generates electrical signals which are correlated to the frequency and amplitude of vibration. These signals are transmitted to the processor on the circuit board 23 for processing.

FIG. 3a illustrates five traces of a typical acoustic ‘signature’ for dispensation of a full metered dose. The ‘signature’ comprises the combination of transient signals produced during the time period covered by the dispensation.

The processor on the integrated circuit board 23 processes and analyses characteristics of the signature including frequency, duration, area, rising slope, falling slope and amplitude of the acoustic signature. The signal processing applied to the signals may include the application of frequency filters, such as notch and/or band-pass filters. In addition amplification of the signals may be applied either across the frequency range or to selected frequencies.

Preferred signal processing regimes are illustrated schematically in FIGS. 4a and 4b. As described above and illustrated in FIG. 3a a typical signature of a full dispensation contains a large number of transient signals of varying frequency and amplitude. It has been found by experiment that for dispensation from a metered dose inhaler the frequency range from 9kHz to 14kHz is particularly important and contains the majority of transient signals created by the dispensation action. As a first signal processing step a band-pass filter is applied to the signal to discard signals having a frequency below 9kHz or above 14kHz. Advantageously, application of the band-pass filter allows the processor to distinguish signatures produced by dispensation from background noise. This significantly reduces the danger of false positive counts where the counter registers a dose as being dispensed due to a background transient noise signature. FIGS. 3b to 3f illustrate typical background noise signatures that may be encountered. FIG. 3b illustrates a typical signature produced by slapping a hand on a table. FIG. 3c illustrates a typical signature produced by tapping the metered dose inhaler on a table. FIG. 3d illustrates a typical signature produced by tapping the pressurised dispensing container of the metered dose inhaler on a table. FIG. 3e illustrates a typical signature produced by stereo music player. FIG. 3f illustrates a typical signature produced by the rattling of keys. In each case it can be seen that the acoustic signatures produced are very different to that produced by dispensation from a metered dose inhaler. In particular, the frequency spectrums are quite different. A further advantage of using a band-pass filter is that the quantity of information passed on to the next stage of signal processing is significantly reduced. This reduces the processing power and electrical power required to process the overall signal.

Next, the processor applies a signature ‘envelope’ to the resultant signal, illustrated schematically in FIG. 4a by numeral 50, wherein upper and lower voltage thresholds are applied to the signal in order to discard very weak or very strong transient signals. In one embodiment, the lower voltage threshold is set at 0.2 Volts and the upper voltage threshold is set at 0.8 Volts. Advantageously, applying a signature envelope to the signal further reduces the degree of subsequent processing that is required.

In the next processing step, shown schematically in FIG. 4b, a notch filter 51 is applied to the resultant signal. The notch filter acts to ‘slice’ the signal into discrete sections of even time duration. In one embodiment the signature is sliced into 10ms slices. The processor then evaluates and counts how many slices contain signals of sufficient strength which fall within the targeted signature envelope.

The memory of the circuit board 23 contains stored information correlated to one or more control acoustic signatures against which the detected signature may be compared by the processor. The control acoustic signatures include one or more signatures produced by correct dispensation of a full metered dose for the particular dispenser 1. Other control acoustic signatures may be included which correlate to partial dispensation of the metered dose.

In the illustrated embodiment, a comparison is made between the number of 10ms slices containing signals in the signal envelope of the detected signature and of the expected number of occurrences from the one or more control signatures. If the number of signal-containing slices in the detected signature matches the expected number then the processor registers this as a full, successfully dispensed dose. To allow for expected tolerances between dispensers, the processor may be programmed to register a full dose where the number of detected slices containing signals is within a predetermined range of the expected number. For example, if the control signature contains 30 slices having signals in the signal envelope then a full dose may be registered where the detected signature contains greater than, say, 27 slices in the signal envelope.

If the number of signal-containing slices in the envelope is less than the expected number, or below the lower tolerance threshold, then a partial dose is registered by the processor.

In an alternative signal processing regime, the overall time duration at which signals are detected within the signal envelope is measured and compared to the time duration of one or more control signals to distinguish between full and partial doses.

In an alternative signal processing regime, the rising slope, falling slope and/or area of the signature may be measured and calculated to distinguish between full and partial doses as well as discriminating actuations from background noise.

Optionally, signatures may also be stored of typical ‘noise’ produced during actuation which is not related to the volume of product dispensed. For example the sliding movement of the pressurises dispensing container will produce noise. This noise may be filtered out of the detected acoustic signature before subsequent signal processing.

The processor, having determined whether a full dose of product was dispensed, can then determine the quantity of product remaining in the pressurised dispensing container 5. The volume of product in a full pressurised dispensing container 5 is pre-programmed into the memory. The volume remaining may then be displayed on the display 27 in the form of an equivalent number of doses. The display may, for example, show the number of doses remaining based on an optimum amount of medicament being dispensed on each actuation, or as a percentage of the amount of medicament in a full container.

Alternative embodiments of the present invention will now be described. Components in the alternative embodiments which are similar to those of the first embodiment have been given like reference numerals and will not be described further except where they differ from the first embodiment.

In a second embodiment, shown in FIG. 5, the integrated circuit board 23, the battery 25 and the display 27 are housed in a clip 29 detachably mounted on the end of the pressurised dispensing container 5 distal from the valve stem 11. The display 27 extends above the housing 3 of the dispenser 1 so that it is visible when the pressurised dispensing container 5 is located in the housing 3.

The acoustic sensor 22 is again mounted proximal to the outlet of the valve stem 11 but in this arrangement is mounted an the distal end of an extension 31 of the circuit board 23 extending along the side of the pressurised dispensing container 5. Preferably, the sensor 22 is located in contact with the ferrule 15 which provides for a good transmission path for acoustic signals. In this embodiment an acoustic chamber is not utilised.

The dose measuring device in accordance with the second embodiment may advantageously be attached to a conventional pressurised dispensing container 5 for use with any unadapted housing 3.

It is envisaged that in an alternative arrangement the dose measuring device may be detachably mounted on the housing of a dispenser.

In a third embodiment, illustrated in FIG. 6, the sensor comprises a microphone 52 affixed to a rear wall 53 of the stem block 16. The microphone 52 detects acoustic signal produced on dispensation and transmits them to the processor.

A fourth embodiment, shown in FIG. 7, is similar to the third embodiment. However, in this embodiment the microphone 52 is affixed to an inner face 54 of the body portion 4 of housing 3. The microphone is therefore directly in the path of emitted sound waves 60 produced from orifice 19 of the stem block 16. This location of the microphone 52 is also advantageous where the display 27 is to be front mounted on the housing 3 since the microphone 52 and circuit board 23 lie in close proximity.

A fifth embodiment, shown in FIG. 8, comprises a sensor for detecting acoustic signals in the form of a piezo-electric element 55 which spans across a bore 56 formed in the stem block 16. The piezo-electric element 55 is directly contacted by product as it is dispensed from the valve stem 11 of the pressurised dispensing container 5. The product is dispensed at high velocity which causes particle or droplets of the product to impact the piezo-electric element 55 before exiting through the orifice 19. Thus, the piezo-electric element 55 acts as a ‘drum-skin’ by flexing and vibrating in a manner correlated to the quantity and duration of dispensed product. The signals thus produced by the sensor 55 are transmitted to the circuit board via wired connections 57.

In a sixth embodiment, shown in FIG. 9, the sensor takes the form of a piezo-electric cantilever 58 which projects into the bore of the stem block 16. During dispensation of product the cantilever 58 is impacted directly by product which cause the cantilever to bend resulting in the generation of electrical signals which are transmitted to the circuit board via wired connections 59.

Whilst the metered dose inhaler has been described in the above embodiments as manually operated the concepts of the present invention may equally be applied to metered dose inhalers that comprise automatic or semi-automatic means of actuation, such as breath-actuated trigger mechanisms.

Whilst the invention has been described by way of example applied to a dispenser in the form of a metered dose inhaler it may equally be applied to other dispensers required to dispense doses of known volumes and which utilise a pressure differential in order to discharge product. Examples include nasal pumps and nebulisers.

The product dispensed by the dispensers of the present invention typically comprise a propellant constituent. However, the invention finds application with dispensers where a pressure differential is applied to the product by other means such as pumped compression, vibration or electro-static excitation. The product need not be a pharmaceutical-based product. The invention may be utilised to dispense any fluid-based product. In particular the product may comprise components in solution and or suspension.

Whilst the dispenser has been described in the above embodiments as comprising a battery in order to power the processor of the circuit board and the display other power sources may be used. For example, the dispenser may be powered by one or more solar cells deriving power from ambient sunlight. Alternatively, the dispenser may be powered by a wind-up mechanical energy storage device as is known in the art. A further alternative is that the dispenser is powered by means of a kinetic energy conversion device as is known in certain designs of watch. In such a device a rotor bearing a permanent magnet is caused to rotate by movement of the dispenser so as to move through a stator coil which generates electrical current. The electrical energy is stored in a rechargeable cell which is then used to power the dispenser. A further alternative where the sensor is a piezo-electric material is to use the electrical current generated during flexure of the material to charge a rechargeable cell which in turn powers the processor and display means. Piezo-electric material is known to be able to generate voltages in excess of 40 Volts which is adequate to generate sufficient charge to power the dispenser as well as providing signals useable to determine the quantity and quality of doses dispensed.

Whilst the signal processing regime has been described above, by way of example, as using a frequency range of 9kHz to 14 kHz, it will be understood that the frequency range may be adjusted to take into account the placement of the sensor, the sensor type and the design of the acoustic chamber, if any, present in the device. In principle frequencies across the audible range may be used, although as stated the frequency ranges mentioned above have been found to be advantageous for the illustrated embodiments.

1-32. (canceled)

33. A dispenser comprising a housing, a pressure sensor, processing means and a display means, the housing being shaped for receiving, in use, a dispensing container of the type containing medicament and having valve means for dispensing the medicament in metered volume doses, wherein, in use, the pressure sensor is capable of detecting a pressure signature produced on dispensation of medicament from the dispensing container, wherein the pressure sensor is operatively connected to the processing means for relaying signals indicative of the pressure signature for processing by the processing means, the processing means being programmed to analyse said signals and compare said signals against one or more data sets containing data indicative of one or more control pressure signatures, the processing means being programmed to use a result of said comparison to detect the quantity of medicament dispensed compared to an intended volume of the metered dose volume, wherein the pressure sensor is isolated, in use, from contact with the dispensed medicament.

34. A dispenser as claimed in claim 33 wherein the processing means is programmed to output a first signal to the display means when said comparison indicates that the quantity of medicament dispensed substantially matches an intended volume of said metered volume dose to thereby update the display means to reflect that a metered volume dose has been dispensed.

35. A dispenser as claimed in claim 33 wherein the processing means is programmed to produce a second signal when said comparison indicates that the quantity of medicament dispensed is substantially less than an intended volume of said metered volume dose.

36. A dispenser as claimed in claim 35 wherein the second signal contains data indicative of the proportion of the intended volume of said metered volume dose actually dispensed.

37. A dispenser as claimed in claim 35 wherein the second signal, or a derivative thereof, is used to update the display means to reflect that a proportion of a metered volume dose has been dispensed.

38. A dispenser as claimed in claim 35 wherein the second signal, or a derivative thereof, is used to produce an alert to instruct a user to administer a further dose.

39. A dispenser as claimed in claim 35 wherein the processing means contains an accumulated volume variable indicative of the accumulated volume of medicament dispensed by the dispensing container.

40. A dispenser as claimed in claim 39 wherein the second signal, or a derivative thereof, is used to update the accumulated volume variable.

41. A dispenser as claimed in claim 40 wherein the accumulated volume variable is used to update the display means to indicate the quantity of medicament dispensed from the dispensing container and or the quantity of medicament remaining in the dispensing container.

42. A dispenser as claimed in claim 33 wherein the processing means analyses one or more of the frequency, duration and amplitude of the pressure signature.

43. A dispenser as claimed in claim 42 wherein the processing means applies a band-pass filter to the pressure signature.

44. A dispenser as claimed in claim 42 wherein the processing means selects a signature envelope for further signal processing.

45. A dispenser as claimed in claim 42 wherein the processing means applies a notch filter to the pressure signature to slice the signature into discrete segments of equal time duration.

46. A dispenser as claimed in claim 45 wherein the processing means compare the number of signal-containing segments with a control number derived from the one or more data sets.

47. A dispenser as claimed in claim 33 wherein the pressure sensor is an acoustic sensor and the pressure signature is an acoustic signature.

48. A dispenser as claimed in claim 33 wherein the pressure sensor is selected from the group consisting of a vibration sensor, a strain sensor and a compression sensor.

49. A dispenser as claimed in claim 47 wherein the acoustic sensor is a microphone.

50. A dispenser as claimed in claim 49 wherein the microphone is a micro-electro-magnetic microphone.

51. A dispenser as claimed in claim 47 wherein the acoustic sensor comprises piezoelectric material.

52. A dispenser as claimed in claim 33 wherein the pressure sensor is located in acoustic contact with an acoustic chamber.

53. A dispenser as claimed in claim 52 wherein the dispensing container received, in use, in the housing is of the type comprising a valve stem through which the medicament is dispensed, the housing further comprising a stem block for receiving said valve stem, the stem block comprising a conduit for directing medicament dispensed through said valve stem towards an outlet of the dispenser, wherein the acoustic chamber is located in acoustic contact with the conduit.

54. A dispenser as claimed in claim 53 wherein the acoustic chamber is located within the stem block.

55. A dispenser as claimed in claim 53 wherein the pressure sensor is located on an external surface of the stem block.

56. A dispenser as claimed in claim 52 wherein the pressure sensor forms one wall of the acoustic chamber.

57. A dispenser as claimed in claim 33 comprising a pyroelectric sensor for detecting temperature changes within the housing during dispensation of medicament.

58. A dispenser as claimed in claim 57 wherein the pyroelectric sensor is operatively connected to the processing means for relaying signals indicative of the temperature for processing by the processing means.

59. A dispenser as claimed in claim 58 wherein the processing means is programmed to analyse said temperature signals and compare said signals against one or more data sets containing data indicative of one or more control temperature signatures, the processing means being programmed to use a result of said comparison to detect the actuation of the dispensing container.
